(19) United States
(12) Patent Application Publication
(10) Pub. No.: US 2004/0048782 A1
(43) Pub. Date: Mar. 11, 2004

(54) Colloidal suspension of submicronic particles for carrying hydrophilic active principles (insulin) and method for preparing same

(76) Inventor: Nathan Bryson, Millery (FR)

Correspondence Address: James C Lydon, Suite 100, 100 Daingerfield Road, Alexandria, VA, 22314, US

(21) Appl. No.: 10/398,133

(22) PCT Filed: Oct. 1, 2001
(86) PCT No.: PCT/FR01/03028

(30) Foreign Application Priority Data
Oct. 6, 2000 (FR) 0012836

(51) Int. Cl. A61K 38/28; A61K 9/16; A61K 9/50

(52) U.S. Cl. 514/3; 424/490

(57) ABSTRACT

The invention concerns a suspension of particles for carrying hydrophilic active principles (insulin). Said carrier particles are based on a polyethylene glycol/hydrophobic neutral polyminoacid double-block polymer. Said polyethylene glycol/hydrophobic neutral polyaminoacid particles are associated with a hydrophilic active principle (insulin). The invention also concerns, a powdery solid from which the transporting particles are derived and the preparation of said solid and said suspension of transporting particles based on polyethylene glycol/hydrophobic neutral polyminoacid particles and insulin. Said preparation consists in copolymerising N-carboxy-anhydrides of hydrophobic neutral polyminoacid particles, in the presence of N-methyl/pyrrolidone, methanol, and amine-functionalised polyethylene glycol, thereby obtaining polyethylene glycol/hydrophobic neutral polyaminoacids, associating the latter with insulin; precipitating with water so as to obtain the carrier particles; optionally carrying out neutralisation, dialysis, concentration and elimination of water, thereby producing a powdery solid or suspended carrier particles and preparing pharmaceutical specialties.

The present invention relates to the field of carrier particles (CP) that are useful for the administration of active principles (AP). The latter are preferably drugs or nutriments for administration to an animal or human organism by the oral or nasal, vaginal, ocular, subcutaneous, intravenous, intramuscular, intradermal, intraperitoneal, intracerebral, parenteral or other route. In terms of their chemical nature, the AP to which the invention relates more particularly are hydrophilic, for example proteins, glycoproteins, peptides, polysaccharides, lipopolysaccharides or polynucleotides.

The present invention relates more precisely to colloidal suspensions of carrier particles, advantageously of the submicron type, that are based on blocks of hydrophobic polyamino acids and hydrophilic polymers of the polyalkylene glycol (PAG) type, preferably of the polyethylene glycol (PEG) type.

The present invention relates both to bare particles as such, and to carrier systems for hydrophilic AP (insulin), consisting of particles loaded with one or more AP.

The present invention further relates to pulverulent solids comprising these CP.

The invention further relates to methods of preparing said colloidal suspensions of particles loaded with hydrophilic AP (insulin).

The purpose of encapsulating AP in CP is especially to modify their duration of action and/or convey them to the treatment site and/or increase the bioavailability of said AP. Numerous encapsulation techniques have already been proposed. The aim of such techniques is on the one hand to enable the AP to be transported to its site of therapeutic action while at the same time protecting it from the aggressions of the organism (hydrolysis, enzymatic digestion, etc.), and on the other hand to control the release of the AP at its site of action so that the amount available to the organism is maintained at the desired level. The AP involved in these changes in transport and residence in the organism are e.g. proteins, but they can also be products that are quite different from organic molecules of synthetic or natural origin. The review by M. J. HUMPHREY (Delivery system for peptide drugs, edited by S. DAVIS and L. ILLUM, Plenum Press, N.Y. 1986) discusses the problem associated with the improvement of AP bioavailability and the advantage of carrier and controlled release systems.

Of all the materials that can be considered for forming CP, polymers are increasingly widely used because of their intrinsic properties. The specifications sheet which it is desired to obtain for the CP is particularly demanding and comprises the following specifications in particular:

1 The first specification sought for the CP would be that the polymer constituting the CP is biocompatible, capable of elimination (by excretion) and/or biodegradable, and particularly that it is metabolized to products that are non-toxic to the organism. In addition, the biodegradation in the organism should be of a sufficiently short duration.

2 It would be advantageous for the CP to be able to form a stable aqueous suspension without the aid of an organic solvent and/or a surfactant.

3 It would also be desirable for the CP to be sufficiently small to be able to undergo, in suspension in a liquid, a sterilizing filtration with a filter whose pore diameter is less than or equal to 0.2 μm.

4 It is desirable for the CP and the CP-AP systems to be obtainable by a method that is non-denaturing towards the AP.

5 The CP should advantageously make it possible to control the rate of release of the AP.

6 Another important specification would be for the CP-AP systems to be able to constitute excellent injectable drugs. This improved suitability for administration by injection—e.g. intravenous or intramuscular injection—or “injectability” is characterized by:

(i) a reduced injected volume (for a given therapeutic dose), and

(ii) a low viscosity.

These two properties are satisfied when the therapeutic dose of AP is associated with a minimum amount of CP. In other words the CP must have a high AP loading factor.

7 The inherent cost of the CP in an injectable preparation must be reduced and, here again, the CP should have a high AP loading factor. In fact, the small size and high loading factor are major specifications sought for the CP.

8 It is also advantageous if the constituent polymer of the CP does not induce an immune response.

9 For the family of hydrophilic AP, particularly proteins and even more particularly insulin, the CP should be adapted to this family of AP in terms of ease of association and release and in terms of non-denaturing character.

The earlier technical proposals, described below, attempted to meet all these specifications. The earlier proposals (a) to (j) will be mentioned by way of illustration:

(a) U.S. Pat. No. 5,286,495 relates to a method of encapsulation by the vaporization of proteins in the aqueous phase with the aid of materials carrying opposite charges, namely alginate (negatively charged) and polylysine (positively charged). This manufacturing process makes it possible to produce particles with a size above 35 μm.

(b) Furthermore, emulsion techniques are commonly used to prepare microparticles loaded with AP. For example, patent applications WO 91/06286, WO 91/06287 and WO 89/08449 disclose such emulsion techniques in which organic solvents are used to solubilize polymers, for example of the polylactic type. However, it has been found that the solvents can be denaturing, especially towards peptide or polypeptide AP.

(c) Biocompatible CP called proteinoids are also known, having been described since 1970 by X. FOX and K. DOSE in “Molecular evolution and the origin of life”, published by Marcel DEKKER Inc. (1977). Thus patent application WO 88/01213 proposes a system based on a mixture of synthetic polypeptides whose solubility depends on the pH. To obtain the matrix microparticles according to said invention, the authors solubilize the mixture of polypeptides and then, with a pH change, they cause proteinoid particles to precipitate. When the precipitation takes place in the presence of an AP, the latter is encapsulated in the particle.

(d) U.S. Pat. No. 4,351,337, which relates to a different field from that of AP transport peculiar to the present invention, may also be cited as a matter of interest. Said patent discloses mass implants fixed and localized in very specific places in the organism. These implants are tubes or hollow capsules of microscopic size (160 μm, with a length of 2000 μm) which consist of polyamino acid copolymers—e.g. poly(glutamic acid/leucine) or poly(benzyl glutamate/leucine) copolymers—obtained by the copolymerization of amino acid N-carboxy anhydride (NCA) monomers. An AP is included by a technique of solvent evaporation from a mixture of polymer and AP. U.S. Pat. No. 4,450,150 belongs to the same family as U.S. Pat. No. 4,351,337 studied above, and has essentially the same subject matter. The constituent PAA are poly(glutamic acid/ethyl glutamate) copolymers.

(e) PCT/FR patent application WO 97/02810 discloses a composition for the controlled release of active principles which comprises a plurality of lamellar particles of a biodegradable polymer that is at least partially crystalline (lactic acid polymer) and an AP absorbed on said particles. In this case the active principle is released by desorption.

(f) The publication “CHEMISTRY LETTERS 1995, 707, AKIYOSHI et al.” relates to the stabilization of insulin by supramolecular complexation with polysaccharides rendered hydrophobic by grafting with cholesterol.

(g) The article published in “MACROMOLECULES 1997, 30, 4013-4017” describes copolymers composed of a polypeptide block based on L-phenylalanine, (benzyl-L-glutamate or O-(tetra-O-acetyl-D-glucopyranosyl)-L-serine, and a synthetic block such as poly(2-methyl-2-oxazoline) or poly(2-phenyl-2-oxazoline). Polymers aggregate in an aqueous medium to form 400 nm particles capable of associating with the enzyme lipase. The term “associated” denotes here that the protein is adsorbed on the particle by a physical phenomenon (no covalent bonding).

(h) PCT patent application WO 96/29991 relates to particles of polyamino acids that are useful for carrying AP, including especially hydrophilic AP such as insulin. These particles have a size of between 10 and 500 nm. The particles according to WO 96/29991 form spontaneously when PAA are brought into contact with an aqueous solution. The PAA comprise hydrophobic neutral amino acid monomers, AAO, and hydrophilic ionizable monomers, AAI.

These particles can be loaded with insulin, preferably in an amount of 6.5% by dry weight of insulin, based on the weight of PAA. Ta is measured by a procedure Ma described below.

(i) EP 0 583 955 discloses polymer micelles capable of physically trapping hydrophobic AP. These micelles consist of PEG/poly-AANO block copolymers (AANO=Amino-Acide Neutre hydrophObe=hydrophobic neutral amino acid).

The AANO can be Leu, Val, Phe, Bz-O-Glu or Bz-O-Asp, the latter being preferred. The hydrophobic active principles, AP, trapped in these PEG/poly-AANO micelles are e.g. adriamycin, indomethacin, daunomycin, methotrexate and mitomycin.

The only examples given in said patent application are micelles based on PEG/polyGlu-O-Bz. Now, it is pointed out that these esters Glu-O-Bz are not stable to hydrolysis in aqueous media. Moreover, said document makes no reference at all to particles consisting of a PEG/poly-AANO block copolymer whose core is formed of the hydrophobic neutral polyamino acid, and comprising a hydrophilic external lattice based on PEG, these particles being capable of associating with hydrophilic AP and releasing them in vivo.

(j) Carrier nanoparticles to which PEG chains are grafted are also known, an example being nanoparticles of polylactides or liposomes. This coating with PEG chains is an effective means, known to those skilled in the art, of avoiding the adsorption of proteins (hydrophilic) on these nanoparticles coated with PEG. The term used to describe such nanoparticles or liposomes is “stealth”. Prevention of the adsorption of proteins on a surface by grafting with PEG is described in a very large number of articles, for example: L. Illum et al., J. Pharm. Sci. 72, 1086 (1983). A description of “stealth liposomes” can be found in D. D. Lasic, F. J. Martin, “Stealth liposomes”, CRC Press (BocaRaton, Fla.) 1995; M. C. Woodle, D. D. Lasic, “Sterically stabilized liposomes”, Biochim. Biophys. Acta 1992, 1113, 171-199; M. C. Woodle, “Controlling liposome blood clearance by surface grafted polymers”, Advanced Drug Delivery Reviews 1998, 32, 139-152.

A summary of these questions may also be found in “Polyethylene glycolcoated biodegradable nanospheres, R. Gref et al., in “Microparticulates for the delivery of proteins and vaccines”, S. Cohen et al., published by Marcel Dekker 1996”. As these stealth nanoparticles are prevented from being loaded with active principle, these authors recommend that the active principles be encapsulated in the core by a method using organic solvent. Now, this type of method does not comply with specifications 2 & 4 of the specifications sheet defined above.

It is therefore apparent from the foregoing that the earlier technical proposals described above, especially proposal (i), incompletely meet the specifications of the specifications sheet indicated above, particularly as regards the association of the particles with hydrophilic active principles (proteins such as insulin) and the ability of these particles loaded with hydrophilic AP to release the latter in vivo without their having been adversely affected by transport.

With these facts established, one of the essential objectives is to be able to provide novel CP which form stable aqueous suspensions of CP spontaneously, without the aid of surfactants or organic solvents, and are suitable for carrying hydrophilic AP (especially proteins such as insulin). The aim is to obtain suspensions of particles loaded with hydrophilic active principle, preferably with proteins such as insulin.

Another essential objective of the present invention is to provide novel CP in stable colloidal aqueous suspension (stable particularly to hydrolysis) or in pulverulent form, based on polyamino acids (PAA), these novel CP preferably meeting specifications 1 to 9 of the specifications sheet referred to above.

Another essential objective of the invention is to improve the particles disclosed in patent application EP 0 583 955.

Another essential objective of the invention is to provide a novel suspension of CP whose characteristics are perfectly controlled, especially in terms of the AP loading factor and in terms of control of the AP release kinetics.

Another essential objective of the invention is to provide injectable hydrophilic medicinal suspensions. The specifications required for such suspensions are a small injection volume and a low viscosity. It is important that the mass of colloidal particles per injection dose be as small as possible, without limiting the amount of active principle, AP, transported by these particles, so as not to detract from the therapeutic efficacy.

Another essential objective of the invention is to provide a colloidal aqueous suspension or a pulverulent solid which comprises active principle carrier particles meeting the specifications referred to above, and which constitutes an appropriate galenical form suitable for administration, for example orally, to humans or animals.

Another essential objective of the invention is to provide a colloidal suspension comprising active principle carrier particles that can be filtered on 0.2 μm filters for sterilization purposes.

Another essential objective of the invention is to propose a method of preparing PAA particles (dry or in suspension in a liquid) that are useful especially as carriers for hydrophilic active principles (especially proteins such as insulin), said method being simpler to carry out and non-denaturing towards the active principles and additionally always allowing fine control over the mean size of the particles obtained.

Another essential objective of the invention is to use the above-mentioned particles, in aqueous suspension or in solid form, for the preparation of drugs (e.g. vaccines), especially for oral, nasal, vaginal, ocular, subcutaneous, intravenous, intramuscular, intradermal, intraperitoneal, intracerebral or parenteral administration, it being possible in particular for the hydrophilic active principles of these drugs to be proteins, glycoproteins, peptides, polysaccharides, lipopolysaccharides, oligonucleotides and polynucleotides.

Another objective of the present invention is to provide a drug, of the type consisting of a system for the prolonged release of active principles, which is easy and economic to produce and which is also biocompatible and capable of assuring a very high level of bioavailability of the AP.

The product-related objectives (among others) are achieved by the present invention, which relates first and foremost to a colloidal suspension of submicron particles capable of being used especially for carrying one or more active principles (AP), these particles being individualized supramolecular arrangements that are:

based on an amphiphilic copolymer comprising:

at least one block of hydrophobic linear polyamino acid(s) (PAA) having α-peptide linkages, the hydrophobic amino acids, AAO, constituting this PAA block being identical to or different from one another;

and at least one block of hydrophilic polymer(s) of the polyalkylene glycol (PAG) type, preferably of the polyethylene glycol (PEG) type;

and capable of associating with at least one AP in colloidal suspension, in the undissolved state, and releasing it, especially in vivo, in a prolonged and/or delayed manner,

characterized in that the particles it contains are associated and/or can be associated with at least one AP selected from hydrophilic AP, preferably proteins, this AP consisting particularly preferably of insulin.

One of the main inventive aspects of these novel carrier particles, CP, in stable colloidal aqueous suspension or in the form of a pulverulent solid, concerns the novel selection of a hydrophilic polymer/hydrophobic polyamino acid block copolymer for obtaining particles of submicron size which form a stable colloidal aqueous suspension in the absence of surfactants or solvents, and which are suitable for hydrophilic AP.

It is particularly surprising and unexpected that particles based on polyalkylene glycol hydrophilic polymer/hydrophobic polyamino acid block copolymer, which are known to trap hydrophobic active principles (EP 0 583 955), are capable of associating with hydrophilic AP, particularly proteins such as insulin, and releasing them in vivo.

In addition, being familiar with the use of an external layer of PEG for preventing the adsorption of hydrophilic proteins, those skilled in the art would quite naturally have discarded this solution in favor of the idea of nanoparticles, which by contrast are said to adsorb a large quantity of hydrophilic proteins. Contrary to all expectations, this is not the case at all within the framework of the invention.

The structure of the PAG/poly-AAO block copolymers and the nature of the amino acids AAO are chosen so that:

the polymer chains spontaneously organize themselves into small particles (CP);

the particles form a stable colloidal suspension in water and in a physiological medium;

the CP associate with proteins or other hydrophilic AP in aqueous media by a spontaneous mechanism that is non-denaturing towards the protein; and

the CP release the hydrophilic AP in a physiological medium and, more precisely, in vivo; the release kinetics depend on the nature of the PAG/poly-AAO copolymer which is the precursor of the CP.

Thus, by varying the specific structure of the copolymer, it is possible to control the AP association and release phenomena from the kinetic and quantitative points of view.

Preferably, the PAG corresponds to polyethylene glycol (PEG) or polypropylene glycol (PPG), PEG being particularly preferred.

According to another characteristic of the invention, the PAG—preferably PEG—has a weight-average molecular weight of between 500 and 50,000 D, preferably of between 1000 and 10,000 D and particularly preferably of between 1000 and 5000 D.

Advantageously, the suspension according to the invention is characterized by a loading factor, Ta, of the carrier particles with insulin, expressed in % of the weight of associated insulin relative to the weight of used insulin, and measured by a procedure Ma, Ta being such that:

7≦Ta,

preferably, 8≦Ta≦50,

and particularly preferably, 10≦Ta≦30.

Procedure Ma:

(a) Preparation of an aqueous solution of insulin: Lyophilized human recombinant insulin (Sigma no. 10259) is poured into 0.1 N HCl solution over 5 min at 25° C. This solution is then poured into a phosphate buffer solution, which is finally neutralized by the addition of 0.1 N NaOH. The solution is subsequently left to stand for 30 min at room temperature and then filtered on a 0.8-0.2 μm “acrodisc®” membrane. The weight of insulin is calculated as a function of the desired volume of solution to give a concentration of 60 IU/ml.

(b) Dispersion of the PAA carrier particles to be associated in the insulin solution: The lyophilized CP are added to the insulin solution at a rate of 10 mg CP/ml solution. This mixture is agitated two or three times in a Vortex and then placed in a rocking stirrer at room temperature for 18 hours. The colloidal suspension is then stored at 4° C.

(c) Separation of the free insulin from the associated insulin and assay of the free insulin: The solution containing the insulin and the CP is centrifuged at 60,000 g for 1 hour at 20° C. The supernatant is transferred to tubes fitted with an ultrafiltration membrane (cut-off threshold: 100,000 Da) and centrifuged at 3000 g for 2 hours at 20° C. The insulin in the filtrate is assayed by HPLC.

For a slightly better definition of the copolymers constituting the particles, it may be indicated that they are of the alternate sequence type (blocks).

Thus, in one preferred embodiment of the suspension of CP according to the invention:

the AAO are hydrophobic neutral amino acids, AANO,

the ratio PAG/AANO is >1,

and the absolute length of the PEG block is >2 monomers, preferably >10 monomers and particularly preferably >20 monomers.

Advantageously, the PAA block(s) based on AANO comprise at least 5, preferably at least 10 and particularly preferably between at least 10 and 50 AANO.

Even more preferably, the particles are “di-blocks” of PEG/AANO.

In practice, these hydrophobic neutral amino acids (AANO) are are selected from the group comprising:

natural neutral amino acids: Leu, Ile, Val, Ala, Pro, Phe, mixtures thereof,

rare or synthetic neutral amino acids: norleucine, norvaline,

and derivatives of polar amino acids: methyl glutamate, ethyl glutamate, benzyl aspartate, N-acetyllysine.

According to one preferred characteristic of the invention, the block PAA constituting the particles have degrees of polymerization, DP, of between 30 and 600, preferably of between 50 and 200 and particularly preferably of between 60 and 150.

The present invention relates not only to suspensions of bare particles as defined above, but also to suspensions of particles comprising at least one active principle, AP. Preferably, the suspension according to the invention is aqueous and stable. These particles, whether loaded with AP or not, are advantageously in a form dispersed in a liquid (suspension), preferably an aqueous liquid, but can also be in the form of a pulverulent solid obtained from the suspension of CP as defined above.

It follows from this that the invention relates not only to a colloidal (preferably aqueous) suspension of CP, but also to a pulverulent solid comprising CP which is obtained from the suspension according to the invention.

Another essential object of the invention concerns the preparation of selected particles (as described above), either in the form of a colloidal suspension or in the form of a pulverulent solid. The method of preparation in question consists essentially in synthesizing precursor PAG/poly-AAO copolymers and converting them to structured particles.

More precisely, the method of preparation is first and foremost a method of preparing the above-mentioned pulverulent solid formed of submicron structured particles capable of being used especially for carrying one or more active principles, these particles being discrete supramolecular arrangements that are:

based on an amphiphilic copolymer comprising:

at least one block of hydrophobic linear polyamino acid(s) (PAA) having α-peptide linkages, the hydrophobic amino acids, AAO, constituting this PAA block being identical to or different from one another;

and at least one block of hydrophilic polymer(s) of the polyalkylene glycol (PAG) type, preferably of the polyethylene glycol (PEG) type;

and capable of associating with at least one AP in colloidal suspension, in the undissolved state, and releasing it, especially in vivo, in a prolonged and/or delayed manner.

This method is characterized in that:

1) at least one PAG segment is reacted with at least one PAA segment, each comprising at least one alkylene glycol or amino acid monomer, respectively, and at least one reactive group for the formation of one or more PAA-PAG linkages (preferably amide linkages) to give a PAG/poly-AAO block copolymer;

2) the PAG/poly-AAO block copolymer obtained in step 1 is precipitated—preferably in water—to result in the spontaneous formation of AP carrier particles;

3) at least one hydrophilic active principle, AP, is associated with the particles;

4) the reaction medium is optionally dialyzed to purify the aqueous suspension of structured particles;

5) this suspension of step 4 is optionally concentrated; and

6) the liquid medium is removed so that the pulverulent solid comprising the particles can be collected.

The functional groups of the PAG and PAA segments of step 1 can be amine or carboxylic acid groups. It is possible to envisage carrying out the polymerization leading to the PAG and/or PAA block before, during or after the formation of the PAG-PAA linkage.

All these variants are within the scope of those skilled in the art. Preferably, in step 1:

1.1) a copolymerization is carried out between monomers formed of amino acid N-carboxy anhydrides (NCA) of hydrophobic amino acids, AAO, in the presence of:

at least one non-aromatic polar solvent preferably selected from the group comprising N-methylpyrrolidone (NMP), dimethylformamide (DMF), dimethyl sulfoxide (DMSO), dimethylacetamide (DMAc) and pyrrolidone, NMP being more particularly preferred;

and optionally at least one cosolvent selected from aprotic solvents (preferably 1,4-dioxane) and/or protic solvents (preferably pyrrolidone) and/or water and/or alcohols, methanol being particularly preferred;

1.2) at least one polyalkylene glycol, PAG (preferably PEG or PPG), polymer block is taken or is prepared by the polymerization of alkylene glycol monomers (preferably ethylene or propylene glycol), this PAG block being functionalized (advantageously at one or both ends) by at least one nucleophilic reactive functional group preferably selected from the group comprising amines (particularly primary or secondary amines), alcohols or thiols; and

1.3) the functionalized PAG of step 2 is added to the poly-AAO block polymerization medium before, during or after the polymerization.

Step 1.1 of the method is based on the known techniques of polymerizing α-amino acid N-carboxy anhydrides (NCA), which are described for example in the article “Biopolymers, 15, 1869 (1976)” and in the work by H. R. KRICHELDORF entitled “α-Amino acid N-carboxy anhydride and related heterocycles”, Springer Verlag (1987).

In one variant, after step 1.1, the poly(AAO/pAAI) copolymer obtained is precipitated—preferably in water—and this precipitate is collected. This variant corresponds to a batch mode of preparing particles in which the poly(AAO/pAAI) copolymer is isolated in the form of a precipitate constituting a stable intermediate. This precipitate can be filtered off, washed and dried, for example.

Particularly preferably, the NCA-pAAI are NCA of O-alkylated glutamic or aspartic acid, for example NCA-Glu-O-Me, NCA-Glu-O-Et or NCA-Glu-O-Bz (Me=methyl—Et=ethyl).

Preferably, the functionalized PAG block(s) is (are) introduced before and/or at the start of the polymerization, which preferably takes place at a temperature of between 20 and 120° C. at normal atmospheric pressure.

Advantageously, the PAG of step 1.2 are commercially available products (e.g. PEG) or are obtained in a manner known per se by the polymerization of ethylene oxide.

Other parameters, such as the polymer concentration, the temperature of the reaction mixture, the mode of addition of the hydrophilic polymer, the use of reduced pressure, the reaction time, etc., are adjusted according to the desired effects well known to those skilled in the art.

The association (step 3) of one or more AP with the particles can be effected by using several methods according to the invention. Non-limiting examples of these methods are listed below.

According to a first method, an AP is associated with the particles by bringing a liquid phase (aqueous or non-aqueous) containing the AP into contact with the colloidal suspension of particles.

According to a second method, the AP is associated with the particles by bringing an AP in the solid state into contact with the colloidal suspension of particles. The solid AP can be e.g. in the form of a lyophilizate, a precipitate or a powder or in another form.

According to a third method, the pulverulent solid (PAA), as described above as a product and by its preparative characteristics, is brought into contact with a liquid phase (aqueous or non-aqueous) containing the AP.

According to a fourth method, the pulverulent solid, as described above as a product and by its preparative characteristics, is brought into contact with the AP in solid form. This mixture of solids is then dispersed in a liquid phase, preferably an aqueous solution.

In all these methods, the AP used can be in the pure form or a preformulated form.

According to optional step 5, the impurities (salts) and the solvent are removed by any appropriate physical separation treatment, for example by diafiltration (dialysis) (step 4), filtration, pH modification, chromatography, etc.

This yields an aqueous suspension of structured particles which can be concentrated, for example by distillation or any other suitable physical means such as ultrafiltration or centrifugation.

To concentrate the particles (step 6) or separate them from their liquid suspension medium (step 7), the aqueous phase is optionally removed, for example by distillation, drying (e.g. in an oven), lyophilization or any other suitable physical means such as ultrafiltration or centrifugation. A white pulverulent solid is recovered at the end of this step 7.

It is pointed out that the implementation of steps 1, 2, 3, 4 and optionally 5 of the above method, corresponds to a preparation of a colloidal suspension of submicron particles with a high hydrophilic AP loading factor.

In this preparation of a colloidal suspension, the PAG/poly-AAO amphiphilic copolymers of step 1 are placed in an aqueous medium in which at least part of the PAG is soluble and at least part of the AANO is insoluble. The PAG/poly-AANO copolymers exist in the form of nanoparticles in this aqueous medium.

An alternative preparation of the suspension of CP according to the invention consists in bringing the pulverulent solid, as described above as a product and by its method of preparation, into contact with an aqueous medium that is a non-solvent for the AANO.

Given the nanometric size of the particles, the suspension can be filtered on sterilization filters, enabling sterile injectable medicinal liquids to be obtained easily and at lower cost. The ability, afforded by the invention, to control the particle size and reach Dh values of between 25 and 100 nm is an important asset.

The present invention further relates to novel intermediates of the method described above, characterized in that they consist of PAG/poly-AAO copolymers that are particle precursors.

According to another of its features, the invention relates to a suspension and/or a pulverulent solid as defined above and/or as obtained by the method described above, this suspension and this solid comprising at least one hydrophilic active principle preferably selected from:

vaccines;

proteins and/or peptides, among which the following are more preferably selected: hemoglobins, cytochromes, albumins, interferons, antigens, antibodies, erythropoietin, insulin, growth hormones, factors VIII and IX, interleukins or mixtures thereof, and hemopoiesis-stimulating factors;

polysaccharides, heparin being more particularly selected;

nucleic acids and preferably RNA and/or DNA oligonucleotides;

non-peptido-protein molecules belonging to various anticancer chemotherapy categories, particularly anthracyclines and taxoids;

and mixtures thereof.

Finally, the invention relates to a pharmaceutical, nutritional, plant health or cosmetic proprietary product, characterized in that it contains a suspension and/or pulverulent solid as defined above, loaded with a hydrophilic AP.

According to another of its objects, the invention further relates to the use of these CP (in suspension or in solid form), loaded with AP, for the manufacture of drugs of the type consisting of systems for the controlled release of AP.

Examples of drugs are those that can preferably be administered by the oral, nasal, vaginal, ocular, subcutaneous, intravenous, intramuscular, intradermal, intraperitoneal, intracerebral or parenteral route.

Examples of cosmetic applications that can be considered are compositions which comprise an AP associated with the CP according to the invention and which can be administered transdermally.

The Examples which follow, relating to the hydrophilic AP formed of insulin, will provide a better understanding of the invention according to its different product/method/application features. These Examples illustrate the preparation of particles of polyamino acids which may or may not be loaded with insulin, and also present the structural characteristics and the properties of these particles.

Figure Captions

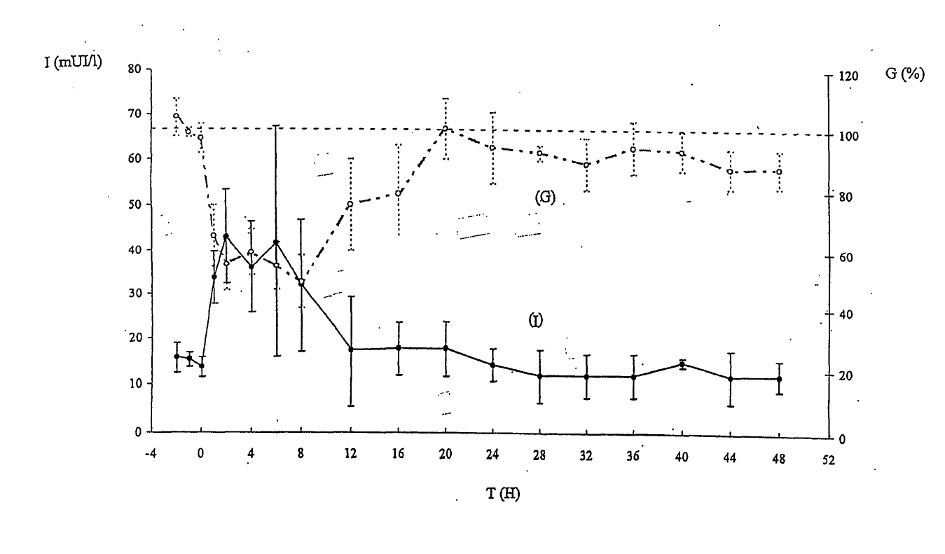

FIG. 1-Change in the glycemia G: [••-o••-] (mean in % relative to basal level) and in the mean insulinemia I (in mIU/l): [—●—] after the injection of a formulation of CP loaded with insulin at a rate of 0.5 IU/kg, as a function of time T (in hours).

### EXAMPLES

### Example 1

Preparation of poly(leucine/ethylene Glycol) Block Copolymer

The techniques used to polymerize NCA to polymers with block or random structures are known to those skilled in the art and are described in detail in the work by H. R. KRICHELDORF entitled “α-Amino acid N-carboxy anhydrides and related heterocycles”, Springer Verlag (1987). The synthesis of one such polymer is specified below.

Synthesis of poly(Leu)₄₀-PEG: 10 g of NCA-Leu are solubilized in 150 ml of NMP at 60° C. 5 ml of a solution of 2 g of aminoethyl-PEG (Mw=5000 D) in 50 ml of NMP are added all at once to the monomer. After 2 h the reaction medium is poured into 1 l of water. The precipitate formed is filtered off, washed and dried. Yield>95%.

### Example 2

Preparation of poly(phenylalanine/ethylene Glycol) Block Copolymer

Synthesis of poly(Phe)₄₀-PEG: 10 g of NCA-Phe are solubilized in 150 ml of NMP at 60° C. 5 ml of a solution of 2 g of aminoethyl-PEG (Mw=5000 D) in 50 ml of N-methylpyrrolidone (NMP) are added all at once to the monomer. After 2 h the reaction medium is poured into 1 l of water. The precipitate formed is filtered off, washed and dried. Yield>95%.

### Example 3

Demonstration of Nanoparticles by Light Scattering (LS) and Transmission Electron Microscopy (TEM)

10 mg of particles of polymer 1 are suspended in 10 ml of water or an aqueous solution of salt. This solution is then introduced into a Coulter granulometer (or laser diffractometer). The results of particle size analysis of the different products tested are shown in Table 1 below.

TABLE 1
Measurement of the size of the CP

| Example | Polymer | Size (nm) |
|---|---|---|
| 1 | poly(Leu)₄₀-PEG | 100 |
| 2 | poly(Phe)₄₀-PEG | 100 |

### Example 4

Test of Association of Nanoparticles with a Protein (Insulin)

An isotonic phosphate buffer solution of pH 7.4 is used to prepare a solution of human insulin containing 1.4 mg/ml, corresponding to 40 IU/ml. 10 mg of the CP prepared in Example 1 are dispersed in 1 ml of this insulin solution. After 15 hours of incubation at room temperature, the insulin associated with the CP and the free insulin are separated by centrifugation (60,000 g, 1 hour) and ultrafiltration (filtration threshold: 300,000 D). The free insulin recovered from the filtrate is assayed by HPLC or ELISA and the amount of associated insulin is deduced by difference. The amount of insulin associated with the CP is greater than 0.77 mg, representing more than 55% of the total insulin used.

The Table below collates the results of the measurements of degree of association performed on different CP. The degree of association expresses the percentage of associated insulin relative to the insulin used in a preparation containing 1.4 mg/ml of insulin and 10 mg/ml of CP. This value is converted to a loading factor which expresses a formulation with 100% protein binding, in mg of insulin per 100 mg of CP.

TABLE 2
Measurement of the degree of association with insulin for a mixture
of 0.14 mg INSULIN/mg CP

| | | Loading factor |
|---|---|---|
| Example | Polymer | mg/100 mg CP |
| 1 | poly(Leu)₄₀-PEG | 13.6 |
| 2 | poly(Phe)₄₀-PEG | >15 |

### Example 5

Pharmacokinetics and Pharmacodynamics of Insulin-Loaded CP in Fasted Healthy Dogs

The protocol of this Example is as follows:

The preparation of Example 4 was injected into dogs which had been rendered diabetic by total pancreatectomy and fasted since the previous evening. At 11 am the preparation was administered subcutaneously into the thorax at a dose of 0.5 IU of insulin per kg of live weight of the animal. The volume administered is between 0.18 and 0.24 ml. At the times −4, −2, 0, 1, 2, 4, 6, 8, 12, 16, 20, 24, 28, 32, 36, 40, 44 and 48 hours, 1 ml of blood is taken by jugular puncture under vacuum into a sodium heparinate tube. 30 μl of whole blood are used immediately to measure the glycemia. The tube is then centrifuged, the supernatant is decanted and the plasma is stored at −20° C. to assay the insulin. The results shown in FIG. 1 below show that the insulin is released up to 12 hours (solid line) and that there is a substantial hypoglycemic effect extending up to 20 hours (broken line) after injection.

TABLE 3
Measurement of the duration of action of insulin (hypoglycemic effect) in
the presence of CP according to the invention

| | | Time to return to |
|---|---|---|
| Example | Polymer | basal level (h) |
| | soluble insulin (without CP) | 1 |
| 1 | poly(Leu)₄₀-PEG | 20 |
| 2 | poly(Phe)₄₀-PEG | >20 |

This Example demonstrates the non-denaturation of insulin in the presence of CP according to the invention.

It also demonstrates the increase in the duration of action of insulin compared with non-formulated insulin, and hence the usefulness of the CP as a system for the controlled release of insulin.

1. Colloidal suspension of submicron particles capable of being used especially for carrying one or more active principles (AP), these particles being individualized supramolecular arrangements that are:
based on an amphiphilic copolymer comprising:
at least one block of hydrophobic linear polyamino acid(s) (PAA) having α-peptide linkages, the hydrophobic amino acids, AAO, constituting this PAA block being identical to or different from one another;
and at least one block of hydrophilic polymer(s) of the polyalkylene glycol (PAG) type, preferably of the polyethylene glycol (PEG) type;
and capable of associating with at least one AP in colloidal suspension, in the undissolved state, and releasing it, especially in vivo, in a prolonged and/or delayed manner,
characterized in that the particles it contains are associated and/or can be associated with at least one AP selected from hydrophilic AP, preferably proteins, this AP consisting particularly preferably of insulin.

2. Suspension according to claim 1, characterized by a loading factor, Ta, of the carrier particles with insulin, expressed in % of the weight of associated insulin relative to the weight of used insulin, and measured by a procedure Ma, Ta being such that:
7≦Ta,
preferably, 8≦Ta≦50,
and particularly preferably, 10≦Ta≦30.

3. Suspension according to claim 1 or 2, characterized in that the PAG—preferably PEG—has a weight-average molecular weight of between 500 and 50,000 D, preferably of between 1000 and 10,000 D and particularly preferably of between 1000 and 5000 D.

4. Suspension according to any one of claims 1 to 3, characterized in that:
the AAO are hydrophobic neutral amino acids, AANO,
the ratio PAG/AANO is >1,
and the absolute length of the PEG block is >2 monomers, preferably >10 monomers and particularly preferably >20 monomers.

5. Suspension according to any one of claims 1 to 4, characterized in that the PAA block(s) based on AANO comprise at least 5, preferably at least 10 and particularly preferably between at least 10 and 50 AANO.

6. Suspension according to any one of claims 1 to 5, characterized in that the particles are PEG/AANO “di-blocks”.

7. Suspension according to any one of claims 1 to 6, characterized in that the AANO are selected from the group comprising:
natural neutral amino acids: Leu, Ile, Val, Ala, Pro, Phe, mixtures thereof,
rare or synthetic neutral amino acids: norleucine, norvaline,
and derivatives of polar amino acids: methyl glutamate, ethyl glutamate, benzyl aspartate, N-acetyllysine.

8. Suspension according to any one of claims 1 to 7, characterized in that it is aqueous and stable.

9. Pulverulent solid, characterized in that it is obtained from the suspension according to any one of claims 1 to 8.

10. Method of preparing the pulverulent solid according to claim 9, characterized in that:
1) at least one PAG segment is reacted with at least one PAA segment, each comprising at least one alkylene glycol or amino acid monomer, respectively, and at least one reactive group for the formation of one or more PAA-PAG linkages (preferably amide linkages) to give a PAG/poly-AAO block copolymer;
2) the PAG/poly-AAO block copolymer obtained in step 1 is precipitated—preferably in water—to result in the spontaneous formation of AP carrier particles;
3) at least one hydrophilic active principle, AP, is associated with the particles;
4) the reaction medium is optionally dialyzed to purify the aqueous suspension of structured particles;
5) this suspension of step 4 is optionally concentrated; and
6) the liquid medium is removed so that the pulverulent solid comprising the particles can be collected.

11. Method according to claim 10, characterized in that, in step 1:
1.1) a copolymerization is carried out between monomers formed of amino acid N-carboxy anhydrides (NCA) of hydrophobic amino acids, AAO, in the presence of:
at least one non-aromatic polar solvent preferably selected from the group comprising N-methylpyrrolidone (NMP), dimethylformamide (DMF), dimethyl sulfoxide (DMSO), dimethylacetamide (DMAc) and pyrrolidone, NMP being more particularly preferred;
and optionally at least one cosolvent selected from aprotic solvents (preferably 1,4-dioxane) and/or protic solvents (preferably pyrrolidone) and/or water and/or alcohols, methanol being particularly preferred;
1.2) at least one polyalkylene glycol, PAG (preferably PEG or PPG), polymer block is taken or is prepared by the polymerization of alkylene glycol monomers (preferably ethylene or propylene glycol), this PAG block being functionalized (advantageously at one or both ends) by at least one nucleophilic reactive functional group preferably selected from the group comprising amines (particularly primary or secondary amines), alcohols or thiols; and
1.3) the functionalized PAG of step 2 is added to the poly-AAO block polymerization medium before, during or after the polymerization.

12. Method according to claim 11, characterized in that the functionalized PAG block(s) is (are) introduced before and/or at the start of the polymerization, which preferably takes place at a temperature of between 20 and 120° C. at normal atmospheric pressure.

13. Method of preparing the suspension according to any one of claims 1 to 8, characterized in that the pulverulent solid according to claim 9 and/or the pulverulent solid obtained by the method according to claim 10 are brought into contact with an aqueous medium that is a non-solvent for the AAO.

14. Method of preparing the suspension according to any one of claims 1 to 8, characterized in that it comprises steps 1, 2, 3, 4 and optionally 5 of the method according to claim 10.

15. Method of preparing the suspension according to any one of claims 1 to 8, characterized in that the hydrophilic AP is associated with the particles by bringing a liquid phase containing said hydrophilic AP into contact with the colloidal suspension of particles.

16. Method of preparing the suspension according to any one of claims 1 to 8, characterized in that the hydrophilic AP is associated with the particles by bringing said AP in the solid state into contact with the colloidal suspension of particles.

17. Method of preparing the suspension according to any one of claims 1 to 8, characterized in that the pulverulent solid according to claim 9 and/or the pulverulent solid obtained by the method according to claim 10 are brought into contact with a liquid phase containing the hydrophilic AP.

18. Method of preparing the suspension according to any one of claims 1 to 8, characterized in that the pulverulent solid according to claim 9 and/or the pulverulent solid obtained by the method according to claim 10 are brought into contact with the hydrophilic AP in solid form, and in that this mixture of solids is dispersed in a liquid phase, preferably an aqueous solution.

19. Intermediates of the method according to claim 10 or 11, characterized in that they consist of PAG/poly-AAO copolymers, preferably PEG/poly-AANO copolymers, that are precursors of particles.

20. Suspension according to any one of claims 1 to 8 and/or obtained by the method according to any one of claims 10 to 18, and/or pulverulent solid according to claim 9, comprising at least one hydrophilic active principle preferably selected from:
vaccines;
proteins and/or peptides, among which the following are more preferably selected: hemoglobins, cytochromes, albumins, interferons, antigens, antibodies, erythropoietin, insulin, growth hormones, factors VIII and IX, interleukins or mixtures thereof, and hemopoiesis-stimulating factors;
polysaccharides, heparin being more particularly selected;
nucleic acids and preferably RNA and/or DNA oligonucleotides;
non-peptido-protein molecules belonging to various anticancer chemotherapy categories, particularly anthracyclines and taxoids;
and mixtures thereof.

21. Pharmaceutical, nutritional, plant health or cosmetic proprietary product, characterized in that it contains a suspension according to any one of claims 1 to 8 and/or obtained by the method according to any one of claims 10 to 18, and/or pulverulent solid according to claim 9.
